(19) United States
(12) Patent Application Publication
(10) Pub. No.: US 2024/0401269 A1
(43) Pub. Date: Dec. 5, 2024

(54) THERMAL-PRESSURE HYDROLYSIS SUSTAINABLE BIOMASS FOR THE PRODUCTION OF ALTERNATIVE PROTEINS AND BIO-MATERIALS

(71) Applicant: THERMO PRESSURE TECHNOLOGIES LIMITED, Dunlin (IE)

(72) Inventor: Andrew Walsh, Whitegate Co. Cork (IE)

(73) Assignee: THERMO PRESSURE TECHNOLOGIES LEMITED, Dublin (IE)

(21) Appl. No.: 18/730,098

(22) PCT Filed: Dec. 21, 2022
(86) PCT No.: PCT/EP2022/087063

§ 371 Date: Jul. 18, 2024

(30) Foreign Application Priority Data
Dec. 21, 2021 (GB) 2118606.9

(51) Int. Cl. D21B 1/36; C12P 19/02; D21C 1/02; D21C 11/00

(52) U.S. Cl. CPC D21B 1/36; C12P 19/02; D21C 1/02; D21C 11/0007; C12P 2201/00; C12P 2203/00

(57) ABSTRACT

The invention provides a fermentable feedstock of sustainable origin comprising a plant biomass thermal pressure hydrolysate, the biomass preferably being lignocellulosic. e.g., grass, straw or similar, to be processed alone or in conjunction with conventional starch/sugar containing crops to supplement or replace conventional arable crops in sugar and nutrient production for fermentation processes. The invention also provides a method of producing an incubatable feedstock for aerobic/anaerobic fermentation comprising subjecting plant biomass to thermal pressure hydrolysis in an autoclave using steam and agitation in an alkaline environment to achieve hydrolytic efficiencies of 85-95% at moderate temperatures of 130-150° C. that is superior to the prior art, recovering the hydrolysed product from the autoclave, and providing this as the incubatable feedstock that may involve solid separation and a saccharification phase prior to fermentation. A method for producing a protein, edible biomass, bioplastic or other bio-material product, comprises supplying the above incubatable feedstock or a feedstock produced by the above method to micro-organisms that can give rise to the protein, bioplastic or other bio-material product within a fermentation reactor and recovering the products therefrom. The invention also provides for the generation of biogas from the residual solids to provide energy for the process while stabilizing the residual organics to allow the resultant digestate to fertilize the land used to grow the sustainable plant materials used as inputs to the process.

### FIELD OF THE INVENTION

This invention relates to the sustainable manufacture of an incubatable feedstock for the production of edible protein-containing substances, biomaterials and biofuels, to a method of producing the feedstock and to a method for producing a protein, biomaterial and biofuel products.

### BACKGROUND TO THE INVENTION

In response to pressing climate change, biodiversity and land-use issues, the sustainability and efficiency of modern industrial processes needs to be transformed to drastically reduce emissions while greatly decreasing the reliance on land and other natural resources to support human society. This transformation is vital so that the planet's biodiversity and ecological health can be allowed to recover. Such industrial processes include food and biofuel production and materials manufacture where the sustainability of the inputs to these processes allied to the energy efficiency need to significantly improve.

### Food Production

Conventional food and beverage production is a particular area of focus as regards its sustainability where conventional industrial farming and fishing are associated with the dramatic destruction of natural habitats that has denuded the planet of its biodiversity. These industrial food production systems are also very significant sources of greenhouse gas emissions. Land-based agriculture contributed 17% of global greenhouse gas (GHG) emissions in 2018 https://www.fao.org/3/cb3808en/cb3808en.pdf. When industrial fishing is added, total global food production contributes approx. 20% to total global GHG emissions. Further to this approx. 50% of these food related emissions are attributable to the production of animal-based food products inclusive of the indirect land and resource use required to produce food for the livestock.

In response to these major land use, resource, and emissions challenges, one radical approach is to displace animal production with equivalent or superior food materials derived from modified plant-based nutrients. Central to this move is the microbial production of products equivalent to meat, dairy products and leather. The latter process is referred to as “biomass fermentation” and/or “precision fermentation”. In simple terms microbes such as yeast, bacteria, fungi, and/or algae are grown in bioreactors and are fed plant-based nutrients. The output of the subsequent microbial fermentation are biomass and microbial metabolites that can substitute for in vivo animal products whether these are animal enzymes and hormones as is already well established in the case of rennet and insulin production through to larger scale generation of milk fats and proteins allied to the generation of material with the same nutritional consistency of animal, avian and fish flesh in addition to other animal byproducts. For example, the collagen in leather can also be produced through fermentation with companies such as Modern Meadow [see e.g. EP-A-2831291 (Forgacs), EP-A-3295754 (Marga), EP-A-3473647 (Dai), EP-A-3452644 (Lee), EP-A-3684800 (Dai) and EP-A-3747901 (Purcell)] and Synbio leather (https:/www.synbio.fi/newleather) having developed fungal fermentation processes to replace traditional animal skin extraction via tanneries. A further example is the generation of dairy milk proteins (whey and casein) through fermentation without the cow. Companies such as Perfect Day (Sg mal: Eres Dat.Day) and Change Foods (Chan Daicy) are marketing dairy equivalent products using fermentation. Such bioreactor fermentation processes have the potential to generate animal proteins with a vastly higher efficiency as regards land and resource use.

This more efficient and sustainable fermentation process at industrial scale has the capacity to potentially replace large sections of traditional animal husbandry, associated land-use, and the resultant emissions, where the nutritional, palatability, utility and cost of the fermentation products becomes equivalent, competitive and/or superior to current animal-based products and processes as in the case of milk, egg, meat and leather products etc. However, in order for meaningful substitution to occur, the scale of this developing fermentation industry will still require very large areas of conventional arable land to be set-aside to supply the plant nutrients to develop the fermentation products that will need to be produced at the scale required. In this regard, current biomass and precision fermentation processes rely on primary agricultural production in the form of combinable grains such as wheat and maize and conventional sugar/starch sources such as sugar cane, maize and sugar beet. As has been seen in the case of biofuel production from “energy crops” that has attempted to attain scale, such dependencies on high-starch/sugar yielding grains give rise to acute land-use and food competition issues that will need to be addressed so that this very promising solution is not constrained by similar sustainability issues associated with monocultures of low diversity arable crops that have intrinsic emission issues in themselves. Further to this the new fermentation industry needs to be inherently cost-effective and carbon neutral or preferably carbon negative to ensure effective and widespread deployment.

### Liquid Biofuel and Alcohol Production

As explained above, the production of liquid biofuels, e.g., bioethanol, biomethanol and biodiesel etc. demands large land areas to be taken from nature to support its production. For example, the vast majority of current bioethanol production is produced from corn/maize starch with biodiesel production relying on rape seed oil. This dedicated production competes heavily with other land-uses whether this is human/animal food production and/or biodiversity. This can create local food scarcity and cost spikes as has been experienced with industrial corn-based bioethanol production in North America. The use of more sustainable feedstocks for bioethanol in particular is already subject to considerable research and initial deployment of technologies that extract sugars from non-starch lignocellulosic biomass from plants (structural carbohydrates) to supplement or replace the dependency on the starch and readily soluble sugars within grains and tubers that are relied upon by humans and animals for nutrition. Similarly, alcoholic beverage (ethanol) production also demands large areas of arable land to support production e.g., hops, barley and wheat etc. Improved efficiency in land use through the use of lignocellulosic structural carbohydrates for sustainable sugar production is also required in this sector to protect biodiversity and reduce emissions.

### Biomaterials and Biochemicals Manufacture

As part of the drive to wean human society off fossil fuels, good progress is being made in the electricity sector where cleaner energy sources such as solar and wind infrastructure is effectively substituting fossil fuel use in electricity production. However, society's requirement to move away from materials such as hydrocarbon-based plastics and industrial chemicals is a challenge that cannot be tackled by renewable forms of electrification alone. This is made more critical due to failure of waste management systems in containing waste plastics from entering ecosystems that is in urgent need of resolution. One solution is to replace many hydrocarbon-based plastics and single-use plastics in particular, with biodegradable bio-based products. While this is relatively straightforward for materials such as paper and cardboard, more sophisticated “bio-plastics” such as polylactic acid (PLA) and other emerging biodegradable polymers such as Poly-3-hydroxyalkanoates; (PHA) are entering the market in increasing volumes to address packaging and material challenges that paper, and cardboard cannot address. However, again, the production process for these biopolymers typically also relies on refined sugars as the raw material to produce these bioplastics through fermentation. In this regard, fermentable sugar is the key input to these processes where for example, approx. 1.6 kg of glucose is required to generate 1 kg of PLA. This process involves the fermentation of the sugar by lactic acid bacteria to generate lactic acid that is then polymerized into PLA for bio-plastics production. An example of this process is provided in WO 2004/057008 (Botelo) in which lactic acid is produced from sugar molasses by fermentation with the bacterium Lactobacillus delbrueckii and polymerised to PLA. Accordingly, production of fermented bioplastics is again tied to conventional refining of sugars from the high starch fruiting bodies within conventional arable crops, i.e., grains and tubers. Consequently, the scaling of these plastic replacement solutions will create their own land-use, biodiversity and emissions pressures due to the scale of the monocultures required to displace petroleum plastics that will inevitably lead to questions over the sustainability of these new industrial processes as scale increases.

### Existing Infrastructure

The primary chemical/biochemical building blocks and nutrients required to generate fermented foods and sustainable replacements for animal products, liquid biofuels, bioplastics and other hydrocarbon based industrial chemicals are typically derived from plant-based carbohydrates (starch/sugars), proteins (amino acids/peptides), fats/lipids and to a lesser extent lignocellulose fibre. These are typically extracted from a limited number of plant species and from the grains/tubers of these plants that are easily accessible to conventional extractive equipment that has not changed appreciably in decades if not centuries. For example, conventional milling and cooking of maize, sugar beet and sugar cane that contain high concentrations of sugars and starch is normally deemed sufficient to achieve commercially viable rates of extraction of soluble sugars for the applications referenced. However, these conventional extraction methods are otherwise highly inefficient as very large amounts of structural carbohydrate remain in the lignocellulosic plant residue. For example, <40% of the total carbohydrate present in combinable crops is typically recovered, i.e., the starch and fermentable sugar components in the grains, where the other 60%+ of the structural lignocellulosic carbohydrate present in the straw, root and chafe is typically discarded. In fact, lignocellulosic biomass represents >90% of all solid-state aboveground carbon across all global terrestrial ecosystems. This gross inefficiency has been historically acceptable as this waste lignocellulose can to a limited extent be used as animal fodder, can be ploughed back into the land, or composted to return the nutrients to the soil. Combustion is also possible where the generation of low-grade heat is extracted for use. However, where such combustion of the straw occurs in situ as is often the case in parts of Asia in the case of rice straw, this gives rise to acute air-quality issues and associated carbon emissions. In the future where animal husbandry is targeted for displacement due to its unsustainable land use and carbon intensity issues, animal feed outlets for lignocellulose residues will be deemed unsustainable and/or simply unavailable. The same applies to processes that seek to extract protein and lipids through conventional milling and/or cooking/solvent extraction which are similarly inefficient as regards the yield of food grade nutrients relative to the large lignocellulosic side streams generated.

To date, efforts to move to more sustainable nutrient inputs for the above industrial processes have focused on the biofuel sector which is the more mature of the three sectors identified above. In this regard, it is recognized that more aggressive treatments of lignocellulosic residues allied to the targeting of non-food plants will be essential to improve the process efficiency, sustainability, and land-use efficiencies associated with biofuel production. The key emphasis has been on the improved extraction of sugars, amino acids/peptides and lipids from existing plant materials while seeking to extract these key biochemical building block materials from more sustainable plant species and from the lignocellulosic residues of conventional crops.

The structural lignocellulosic parts of plants are primarily composed of three primary components, i.e., lignin, cellulose and hemicellulose where the levels of starch and readily fermentable sugars are low. The lignin present forms a protective layer over the underlying cellulose and hemicellulose that protects these carbohydrates from enzymatic attack. This is the primary reason for the very low sugar yields when such lignocellulosic materials are subjected to digestive enzyme exposure during digestion by ruminants or cooking as applied in industrial sugar manufacture. Accordingly, the extraction of sugars from these plant parts requires more aggressive chemical breakdown of the recalcitrant lignin to allow the cellulose and hemicellulose to be released. The release of cellulose facilitates its enzymic conversion to glucose, while the released hemicellulose can be enzymatically converted to primarily xylose (among other sugars). Where glucose and xylose can be efficiently extracted from lignocellulose materials on a cost-effective basis, this opens up the possibility to target a much wider array of plant species and crop parts for sugar production other than the limited diversity of conventional starch-based food crop grains and tubers as are currently cultivated and extracted.

The process of cleaving sugars and other nutrients from these structural lignocellulose materials in an aqueous environment is generically referred to as “hydrolysis”. This can be defined as the process of solubilizing organics from insoluble parent materials. Typical lignocellulose materials that have high levels of cellulose and hemicellulose present relative to easily extractable sugars and starches include barley, wheat, oats and rice straw, corn stover in addition to grasses (fresh cut material or hay or silage), green cuttings, leaves, aquatic plants, and seaweeds. Other more sustainable and diverse plant species could also be potentially targeted for food, biofuel and biomaterial production depending on the local climate and land use suitability.

To extract these sugars from the parent recalcitrant lignocellulosic material, more aggressive hydrolytic processes must be applied than are applicable to high starch plant parts where simple cooking in water at <100° C. is typically sufficient. These industrial hydrolytic processes fall into five main categories:
(a) Improved mechanical extraction
(b) Strong acid and alkali treatment
(c) Extrusion
(d) Enzymatic treatment
(e) Thermal treatment

In the case of (a), the consensus is that while improved mechanical treatments such as milling may be a useful as a step in the process, mechanical treatment alone does not directly influence the biochemistry of the lignocellulosic matrix necessary to release the bulk of the cellulose and hemicellulose to facilitate subsequent saccharification of the underlying cellulose and hemicellulose. Accordingly, the saccharification efficiency of mechanically milled lignocellulose is typically just 40-50% and then only after protracted retention times of weeks of enzymatic exposure.

In the case of (b), strong acid/alkali treatment, while effective as a method of improving the hydrolysis/delignification of lignocellulose and the subsequent saccharification, these methods are typically seen as undesirable due to cost, safety, and process management concerns. In the case of acid treatment, Lightner (U.S. Pat. No. 6,258,175) describes an acidic treatment method for the processing of lignocellulose material to facilitate downstream bioethanol production where the lignocellulose material needs to be milled to <1 mm ahead of acid treatment. Holtzapple et al. U.S. Pat. No. 5,171,592 describes an alkaline pre-treatment process again ahead of bioethanol production where calcium hydroxide or calcium oxide is added to the lignocellulosic material and then heated at <100° C. as a method to enhance delignification to release cellulose.

Further to the above examples, there is also extensive literature on the use of alkaline hydrolysis for the pre-treatment of lignocellulose biomass ahead of anaerobic digestion and fermentation at varying concentrations and temperatures where most of this work relates to laboratory bench scale reactors. Sambuisiti et al. (2013) for example describes a method of treating wheat straw with 10% NaOH at 100° C. where the released carbohydrates in the resultant hydrolysate showed a bioavailability to anaerobic microbes of 71%. Therefore, while the hydrolytic efficiencies of acid/alkali extraction are better than mechanical treatment, these do not reach the efficiencies required for widespread commercial deployment.

Extrusion (c) is a composite approach between mechanical treatment (a) and thermal treatment (e) as below. Extrusion involves mechanically applying a very brief high-pressure shear force on the lignocellulose material that raises the temperature of the substrate that partially disrupts the structure of the lignocellulosic material. Extrusion therefore partially removes or perforates the protective lignin layer to allow improved enzymatic access to the underlying cellulose and hemicellulose in subsequent microbial/enzymatic transformation processes. Therefore, while extrusion is a significant improvement on conventional mechanical milling, as the contact times are very short (seconds), extensive degradation of the lignocellulose is not possible. This again only results in a moderate subsequent saccharification efficiency that is similar to chemical/alkali treatments of 50-75%.

In comparison, option (d): direct enzymatic/microbial hydrolysis of lignocellulose is much more established as a method of hydrolysing lignocellulose materials and is being deployed at large scale in the biofuel sector while also being examined as an option for the protein fermentation industry where this is typically preceded by a mechanical milling step. The main disadvantage of the enzymatic approach is the speed where exposure/dwell times within the enzymatic/microbial reactors can be the order of weeks. This process protraction is again due to the presence of the protective lignan layer around the hydrolysable cellulose and hemicellulose that is not removed by mechanical methods, and this greatly slows the microbial/enzymatic saccharification of the lignocelluloses present into soluble sugars. The presence of intact lignin limits the final conversion efficiencies even after extensive exposure times. Such enzymatic/microbial hydrolytic processes therefore require very large reactors that are expensive to manufacture and operate.

Option (e) refers to as thermal treatment and encompasses aqueous processes where the temperatures of the lignocellulose materials are maintained above the boiling point of water, typically 150-220° C. in a pressure vessel. This process is referred to interchangeably as thermal hydrolysis, hydrothermal hydrolysis, thermal pressure hydrolysis and/or liquid hot water treatment. A further version that is commonly applied to lignocellulosic biomass is referred to as “steam explosion” where the pressure applied to the substrate is suddenly released at the end of the steam exposure period in an attempt to further improve the fragmentation of the biomass cellular structure and further separate the lignin from the cellulose and hemicellulose. Such aqueous thermal treatment processes conducted in pressure vessels can therefore be differentiated from conventional cooking as typically applied in sugar production (where this cooking occurs at <100° C. at atmospheric pressure). Such low temperature cooking is sufficient to extract sugars from starch, where little or no lignocellulosic sugars will be released. Various embodiments of thermal treatment technologies have been demonstrated to achieve delignification/saccharification efficiencies in the 70-95% range. However, such improved efficiencies are correlated with increasing severity of processing, i.e., thermal treatments that result in high sugar recovery efficiencies in downstream enzymatic/microbial processes are associated with processes where temperatures of >180° C. are applied, and often >200° C. Examples of this is provided by Larsen et al. (2012) as regards the “Inbicon” process used to generate bioethanol from cellulose recovered from thermally treated straw. Such efficiencies not only come at the price of high energy inputs, but these high temperatures are also associated with the generation of phenolic lignin degradation by-products that are toxic to the downstream microbial processes if not removed which therefore necessitates a separation and washing process to recover the cellulose without the toxic phenolics. This toxicity has also been observed in acidic hydrolysates, and therefore such high temperature pre-processing can ultimately result in lower net yields of sugars and/or more complex processes. Furthermore, where significant concentrations of sugars and starch are present in the plant biomass being processed, such high temperatures can give rise to the caramelization, i.e. a destruction of a significant proportion of these sugars.

The specific configuration of the thermal pressure hydrolysis (TPH) process (as per the current invention) represents a substantive improvement on the prior art where hydrolytic efficiencies of between 80 and 95% are achievable at lower temperatures. Efficiencies in the 80-85% range are associated with the application of pressure and heat alone in a steam environment at moderate temperatures of 140-160° C. This process is acidic where the acid is generated by the release of organic acids during hydrolysis as opposed to the addition of acids to the process. In contrast, the higher efficiency levels in the 85-95% range are associated with the conduct of the TPH process under moderate to strong alkaline conditions (pH 10-14) but at lower temperatures of 130-150° C. The alkaline catalysis of the delignification and hydrolysis of lignocellulose during the TPH process at these lower temperatures allows for maximum downstream sugar yields while minimising the generation of toxic phenolics and loss of sugars due to caramelization. In this regard, any starch present will be fully converted to soluble sugars with heat and pressure alone within the reaction vessel, while the delignification required to release hemicellulose and cellulose can both be expected to achieve close to 90-95% efficiency following catalysed alkaline TPH treatment. Furthermore, the lignin component (that can represent 10-30% of the lignocellulosic biomass being treated), while separated from the cellulose and hemicellulose, will be generally preserved. Accordingly, the released insoluble lignin fibres are also available for biological conversion to useful biochemicals and food components in downstream fermentation reactors in addition to the cellulose and hemicellulose where selected fungal strains are deployed that can metabolise lignin. This process combination will therefore give rise to very high efficiency rates as regards the transformation of total lignocellulosic biomass into final fermented products.

To achieve temperatures above the boiling point in an aqueous suspension of lignocellulosic material, the process must be managed within a pressure vessel. As above, the most common version of the process is referred to as “steam explosion”. Steam explosion, while applied to the biofuel industry for the generation of biofuel (bioethanol) and as part of the “Kraft” paper and cardboard manufacturing process, is not yet in use in the emerging protein fermentation and bioplastics industries.

A number of publications describe the treatment of energy crops and vegetable waste followed by so-called “steam explosion” method in order to make cellular contents accessible to downstream microbial/enzymatic processes. For example, EP 2576757B1 (Dauser) explains that steam explosion is a technical process by which input material is heated up to 300° C., preferably to 150-200° C. at 3-20 bar for a period of time within a static, mixed pressure vessel (hydrolyser), after which the pressure is abruptly returned to atmospheric pressure. This rapid decompression is said to facilitate breakdown of cells, after which the cell contents are available in liquefied form for further processing. However, the Dauser specification also does not provide any details of the hydrolysis efficiency as it relates to the biochemical changes of the plant materials presented to the apparatus. Nor are there any details as to the refinement of the biochemical inputs to the downstream biogas and biofuel processes other than screening out contraries such as rocks. No mention is made as regards its potential use for the preparation of nutrients for protein fermentation, biochemicals or bioplastics production. Similar steam explosion processes are described for the preparation of straw for anaerobic digestion (Bauer et al, 2014). This method demonstrates that the release of cellulose and hemicellulose for microbial metabolism is approx. 73% of the theoretical maximum based on biogas yields from the treated straw when the straw is exposed to 165° C. prior to steam explosion.

Other embodiments of steam explosion equipment used in the biofuel industry are generally similar to the Dauser and Bauer designs as regards the steam explosion hydrolysis reactor design where the target biomass is managed in static pressure vessels and where the input materials are agitated through the use of paddles, screws and other mechanical mixers. These systems are considered to be significantly inefficient as regards optimising the hydrolysis of the lignocellulose materials relative to the invention described herein where the steam explosion processes must be operated at temperature (>160° C.) which are reported to achieve the higher hydrolytic efficiencies demanded by industrial processes and therefore result in the generation of toxic by-products), that necessitate additional intermediate refinement steps to avoid inhibition in the downstream biological reactors as described by Larsen et al. (2012).

The main commercial embodiments of steam explosion technology in commercial use are applied to the hydrolysis of sewage sludge operating typically at 155-165° C. Steam explosion and the associated hydrolysis of sewage sludge is pursued to improve the biogas potential of the sludge, improve its subsequent de-watering characteristics of the sludge allied to improving its sanitary quality through the elimination of pathogens. However, equipment such as the CAMBI process (www.cambi.com) are designed specifically to handle fluids, e.g., sewage sludges and de-contaminated food sludge that can be pumped. These systems cannot process fibrous lignocellulose plant materials and they are not configured to provide refined hydrolysis products to demanding downstream processes such as protein fermentation and bioplastics manufacture. CAMBI did however also obtain a patent for the thermal hydrolysis of coarse biomass (WO 2006/032282). This method, however, is complicated by the requirement for the combination of steam explosion with wet oxidation and the use of strong oxidising agents such as hydrogen peroxide. In any event the enzymatic yield of xylose and cellulose described at <50% and 80% respectively. This then provides a total reducing sugar yield of <70% overall.

U.S. Pat. No. 10,907,303 (Toll et al.), the contents of which are incorporated herein by reference, discloses that low density lignocellulosic material can be treated to allow the effective processing of the large volumes of lignocellulose biomass required to allow inclusion of the hydrolysed product within anaerobic digestion (AD) systems at scale by making the organic content of lignocellulose bio-available to anaerobic microbial degradation. This involves the specific destruction of buoyancy while making the cell contents available for AD by treatment in a pressure vessel with saturated steam but without steam explosion. The process prepares a fibrous primary lignocellulose biomass for AD, which comprises: providing the fibrous primary lignocellulose biomass in a finely divided state; compressing the biomass and adding water and/or organic slurry to the biomass to form a feed batch having a bulk density of >350 kg/m³; introducing the feed batch into a pressure vessel wherein the feed batch contains >125 kg of the biomass per m³ and wherein the pressure vessel is rotary, has inlet and discharge ends provided with inlet and discharge doors and has a downward incline towards its discharge end; introducing an atmosphere of saturated steam into the pressure vessel and maintaining the saturated steam atmosphere within the pressure vessel at 133-220° C. and at 3-10 bar for TPH whilst circulating the biomass through the saturated steam atmosphere within the pressure vessel by helical internal flights of the pressure vessel for a time effective to induce internal collapse of the lignocellulose; gradually depressurizing the pressure vessel to atmospheric pressure or lower and cooling its contents; and recovering a hydrolyzed lignocellulose biomass from the discharge end of the pressure vessel as a slurry or sludge in a sterilized state, with a disrupted cellular structure as a result of the TPH process, and with loss of an inherent buoyancy of the biomass in aqueous liquids. The primary lignocellulose biomass may comprise straw e.g., from barley, oats, rape, rice, rye, and wheat or a mixture thereof. The hydrolysed lignocellulose biomass recovered from the TPH vessel may be combined with a liquor from an anaerobic digestion, water and/or wastewater in a mixing tank and subjected to anaerobic digestion to produce biogas.

### SUMMARY OF THE INVENTION

In one aspect the invention provides a fermentable feedstock comprising a plant biomass thermal pressure hydrolysis (TPH) hydrolysate.

In a further aspect the invention provides a method of producing an incubatable feedstock for fermentation comprising:
subjecting plant biomass to thermal pressure hydrolysis in an autoclave using steam and agitation;
recovering the hydrolysed product from the autoclave; and optionally
removing solids from the hydrolysed product to recover a liquid phase which is a sugar/nutrient solution and provides the incubatable feedstock or utilizing the whole hydrolysate as the incubatable feedstock.

In a preferred aspect, the plant biomass is a fibrous primary lignocellulose biomass which is subjected to fermentation by a method which comprises:
introducing a feed batch into a pressure vessel;
adding water and/or organic slurry to said feed batch;
introducing an atmosphere of saturated steam into the pressure vessel and maintaining said atmosphere at 110-180° C. and 2-8 bar whilst circulating the material of the feed batch through the saturated steam atmosphere for a time effective to induce internal collapse of the lignocellulose biomass;
gradually depressurizing the pressure vessel and cooling its contents; and
recovering the hydrolyzed lignocellulose biomass from the pressure vessel as a slurry/sludge in a sterilized state, with a disrupted cellular structure as a result of thermal pressure hydrolysis.

In a further aspect the invention provides a method of producing an incubatable feedstock for fermentation comprising:
subjecting primarily lignocellulosic plant biomass to thermal pressure hydrolysis in a TPH vessel using steam and agitation as described in U.S. Pat. No. 10,907,303;
recovering the hydrolysed product from the TPH vessel; and
(a) removing solids from the hydrolysed product to recover a liquid phase which is a sugar/nutrient solution and provides the incubatable feedstock or
(b) transferring the whole hydrolysate as the incubatable feedstock to a saccharification reactor followed by the transfer of the resultant sugar solution to downstream fermentation reactors to generate proteins, digestible carbohydrates, lipids, biofuels and/or other biochemicals or
(c) transferring the separated solid dissociated lignin, cellulose and hemicellulose fibres to the saccharification reactor with the liquor fraction being recovered for reuse in the TPH process.

The invention described in U.S. Pat. No. 10,907,303 (Toll) is a process of preparing a fibrous primary lignocellulose biomass for anaerobic digestion, which comprises:
providing the fibrous primary lignocellulose biomass in a finely divided state;
compressing the biomass and adding water and/or organic slurry to the biomass to form a feed batch having a bulk density of >350 kg/m³;
introducing the feed batch into a pressure vessel wherein the feed batch contains >125 kg of the biomass per m³ and wherein the pressure vessel is rotary, has inlet and discharge ends provided with inlet and discharge doors and has a downward incline towards its discharge end;
introducing an atmosphere of saturated steam into the pressure vessel and maintaining the saturated steam atmosphere within the pressure vessel at 133-220° C. and 3-10 bar for thermal pressure hydrolysis whilst circulating the biomass through the saturated steam atmosphere within the pressure vessel by helical internal flights of the pressure vessel for a time effective to induce internal collapse of the lignocellulose;
gradually depressurizing the pressure vessel to atmospheric pressure or lower and cooling its contents; and
recovering a hydrolysed lignocellulose biomass from the discharge end of the pressure vessel as a slurry or sludge in a sterilized state, with a disrupted cellular structure as a result of the thermal pressure hydrolysis and with loss of an inherent buoyancy of the biomass in aqueous liquids. The invention provides a fermentable feedstock comprising a plant biomass thermal pressure hydrolysis (TPH) hydrolysate resulting from the above process.

The present invention contemplates further improvements in the capacity of the TPH vessel through the use of pelletising equipment to improve the bulk density of the straw entering the reactor further to >500 kg/m³ that when mixed with an organic slurry suitable for food production provides a bulk density of the mixture is >600 kg/m³ where this improved density provides for higher biomass throughput per TPH batch and overall improved sustainable sugar yields for the facility where less batches are required for the same lignocellulose biomass input tonnage. In this regard, while the pelletisation of lignocellulosic biomass such as straw and hay has been described ahead of anaerobic digestion (Briquettes for Biogas production worldwide (kineticbiofuel.com), the use of palletisation/briquetting ahead of thermal hydrolysis has not been described.

The variation in the processing of the lignocellulose fibres and the management of the liquor fraction (as described above) relates to the species of plant utilized and/or plant component utilized allied to the downstream fermentation and anaerobic digestion process requirements. The combination of the sequential saccharification and fermentation steps is also possible where these two microbial/enzymatic processes can be conducted in parallel within a single reactor as will be the case in some embodiments.

In an aspect, the invention provides a method of precision and/or biomass fermentation which comprises supplying a fermentable feedstock comprising a plant biomass thermal pressure hydrolysis filtrate, fibre or whole hydrolysate to precision/biomass fermentation reactor or reactors containing enzymes and/or microorganisms selected or genetically modified (provided with instructions in the form of DNA) to produce a target biochemical product (e.g., milk proteins such as whey and casein) and recovering the product from the reactor. Examples of target biochemical product molecules include acetone, isopropanol or a combination thereof, see US 2018/305720 (Tracy et al., White Dog Labs), collagen and fibronectin, particular proteins e.g., chymosin, see EP2216402 (Gonzalez Villa), single cell protein e.g., the protein powder Plentify (White Dog Labs), and recombinant growth factors, etc. The conversion of the xylose released from delignified hemicellulose to the alcohol sugar xylitol and daughter synthesis molecules such as glycols and polyesters is also envisaged.

The invention also provides for the generation of biogas from the residual solids and side-stream residues of the fermentation processes to provide energy for the process while stabilizing the organics to allow the resultant digestate to fertilize the land used to grow the sustainable plant materials used as inputs to the process which is desirable for the sustainability and circularity of the invention within the bioeconomy. The described process therefore provides for the digestion of the residual organics and waste fermentation biomass to generate energy in the form of biogas to provide some or all of the electrical and/or heat energy required for the TPH, fermentation and auxiliary processes. The stabilized digestate product after anaerobic digestate may be used to generate a peat replacement product and/or to recycle nutrients to the lands used to generate the input biomass for the process. A plurality of TPH vessels, saccharification and fermentation reactors and biogas reactors are envisaged for large-scale biorefinery deployment of the technology for the protein and biochemical production levels that will be required to displace and/or substitute for animal agriculture and fossil hydrocarbons.

In another aspect the invention provides a method for producing a high calorific value food product with high human and/or animal digestibility relative to the input lignocellulosic material. The production of bulk protein, lipid and carbohydrate products at scale is envisaged. These nutritional products are generated from the above incubatable feedstock or incubatable feedstock produced by the above method and through the use of a microorganism or microorganisms that can generate the protein/lipid/carbohydrate products within the fermentation reactor(s) and recovering the protein/lipid/carbohydrate product therefrom. The targeting of all primary hydrolysate components such as the xylose, glucose, lignin and protein present for extraction as useful biochemicals and food is envisaged to optimise the overall efficiency of the system compared with first generation systems that typically only focus on the glucose potential of cellulose within the bulk lignocellulose biomass. The above fermentable or incubatable feedstock can therefore also be converted according to the invention into liquid biofuels more efficiently through known chemical and/or biochemical synthesis pathways. Beverages, biomaterials and biochemicals can also be produced with a focus on displacing fossil hydrocarbon inputs e.g., the manufacture of bioplastics such as PLA or PHA and the generation of biomass derived glycols and polyesters.

We have found that the deployment of thermal-pressure hydrolysis (TPH) technology for the purposes of biological waste treatment as described WO 2012/172329 (Toll et al.), has demonstrated that lignocellulosic waste material can be mechanically liquefied and hydrolysed to facilitate pumping and subsequent enhanced biogas production within conventional downstream anaerobic biogas reactors. This patented TPH process has been examined further in relation to the biochemical processes involved during the hydrolysis of lignocellulose materials where it has been improved to the point of 95% efficiency in downstream saccharification reactors where the lignocellulosic carbohydrates present can be saccharified into bioavailable sugars and organic acids given the very efficient delignification achieved. In this regard, and as has been demonstrated at pilot scale, (coupled with successful full-scale performance comparisons), this industrial apparatus is capable of processing 10-30 tonne batches of primary lignocellulose biomass on a fresh weight basis such as grass silage, hay and straw within the standard 64 m³ reactor. Conventional high-starch crops such as sugar beet and corn can be hydrolysed within a timeframe of 2.5-3.5 hours inclusive of reactor filling, pressurization, sterilization, hydrolysis, de-pressurization, and discharge. As described in U.S. Pat. No. 10,907,303 (Toll), this equates to a loading rate of 250-360 kg/m³ of liquid medium that is essential for commercial operation. This bulk density can then be improved further through the pelletisation of low-density lignocellulosic materials such as straw ahead of TPH processing (>500 kg/m³). This is superior to most other hydrolysis/delignification systems that typically operate at <150 kg/m³. Such low loading rates within the prior art results in much larger hydrolysis reactors that demand much higher energy inputs to heat the substantial volumes of water involved to suspend the target lignocellulosic biomass being processed. Moreover, most of these processes described in the literature have not progressed beyond bench-scale for these reasons.

Where catalysed hydrolysis conditions in the TPH vessel as described above are maintained under alkaline conditions within the pressurised steam environment of the rotary vessel, this process results in 85-95% of the cellulose and hemicellulose being released to facilitate downstream microbial/enzymatic conversion of these polymers into glucose and xylose respectively among other sugars. The treatment regime will also result in a proportion of the hemicellulose being converted into xylose within the TPH vessel. Other soluble sugars will be readily released from ruptured cells with a higher extraction efficiency than conventional cooking processes allied to the hydrolysis of the starch present. The proportions will depend on the primary plant species and plant components treated, i.e., higher starch-containing crops such as sugar beet can be expected to generate the higher yields of sugar through enhanced cellular disruption. More lignified materials such as straw and hay will yield relatively lower sugar amounts immediately after the initial TPH pre-treatment stage, where the bulk of the glucose and xylose is released during subsequent enzymatic/microbial saccharification processing of the delignified biomass in the downstream reactors.

From test run comparisons for various plant materials, the improvement in total reducing sugar yields from high starch materials such as sugar beets were found to be 10-15% better than conventional cooking processes (<100° C.). However, in the case of lignocellulose materials with moderate levels of soluble sugar present that have good ruminant digestibility such as grass silage/haylage, the immediate improvement in the release of soluble sugars post-TPH due to cellular disruption has been demonstrated to release 22% of the available organic material as reducing sugars from late season haylage where negligible sugar could be extracted through conventional non-enzymatic aqueous pulping (see below).

The process also releases amino acids from the crude protein present and fatty acids from any lipids present where again the relative proportions of these nutrients present will be plant species and plant component specific. The time/temperature settings to optimise hydrolysis will also depend on the plant biomass species and components being processed. For example, less aggressive treatments (110-120° C.) under mild alkaline conditions are required for fruiting bodies such as sugar beets and grains that contains higher initial proportions of readily extractable sugars and starch. Higher temperature and alkalinity regimes are required to optimise the extraction of sugars from more recalcitrant lignocellulose materials such as first cut grasses, i.e., 130-150° C., with later grass cuts, hay, straw, stover and leaves may require higher temperature treatments of 140-165° C. However, the high efficiency described can be achieved in the lower temperature range when the alkalinity is higher. This matrix of temperature, pressure and alkalinity can therefore be adjusted depending on energy, chemical consumption and downstream microbial considerations to maintain the efficiency of the system in the 85-95% range.

The other important feature of high temperature processing is that all native and contaminant microbes present within the raw biomass whether bacteria, spores, viruses, fungi, or algae will be thermally destroyed. This is important as regards preventing biological contamination of the downstream fermentation biomass populations, i.e., the TPH process is a sterilization process as opposed to a pasteurization technology as is conventionally used in fermentation. This feature therefore provides a higher degree of process protection and biosecurity from unwanted microbes where sterile conditions are demanded to avoid competing or pathogenic growth within fermentation bioreactors that will affect product yields and quality.

As a further observation, there is a very high correlation between the total soluble carbohydrate/sugar yield generated following the saccharification of hydrolysed lignocellulose with the biogas potential of the hydrolysed substrate following TPH treatment. In this regard, the biogas and/or the biomethane yield as determined using the standard biomethane production (BMP) test can be used to determine the enzymatic bioavailability of lignocellulose biomass subjected to various pre-treatment regimes. This correlation between fermentation and BMP performance allows the TPH hydrolysis process to be rapidly assessed as regards its efficiency in promoting down-stream saccharification and associated fermentation. The TPH-BMP assessment of various plant species and lignocellulose types has demonstrated a universal increase in the bioavailability of sugars, organic acids and other nutrients to microbes compared with controls and incumbent hydrolytic processes. The hydrolytic efficiencies of incumbent technologies targeting wheat and barley straw substrates is illustrated in FIG. 3. This illustrates the superior hydrolytic efficiency of the TPH process using heat alone (acidic conditions) or in combination with an alkali relative to the typical yields from commercial hydrolysis technologies. This comparison is based on the BMPs achievable as a function of time versus the theoretical maximum yield of methane from straw. As illustrated using a fixed hydraulic retention time of 30 days, mechanical processes such as milling has a hydrolytic efficiency of <50%. Steam explosion at moderate temperatures (<165° C.) provide an efficiency of approx. 60%. This efficiency is similar to alkaline and acidic treatments conducted below 100° C. Published data suggest that yield efficiencies similar to the TPH or the alkali-TPH as per FIG. 3 are only associated with high temperature processing of 185-205° C. (Aski et al., 2019) that requires significantly higher energy inputs and where caramelization and phenolic toxicity becomes an issue. As can be seen in FIG. 3, heat treatment alone at 155° C. within the TPH vessel yields a hydrolysis efficiency of approx. 80% after 30 days where the alkali catalysed TPH process has an efficiency approaching 95% at a temperature of 140° C.

This very significantly improved hydrolysis efficiency using the TPH process at lower temperatures facilitates the utilization of a wide diversity of plant types allied to being able to harness the complete plant as opposed to the current reliance on the high sugar/starch fruiting bodies/grains and tubers. This in turn provides an opportunity to remove society's reliance on the monoculture of species with low diversity and high land use demand. This feature also provides for significant climate change resilience as regards this significant improvement in biomass productivity.

This invention can therefore play a pivotal role in the three key industries that require large volumes of sustainable nutrients to be presented to the respective microbial reactor systems, whether the outputs are fermented proteins, biofuel or biochemicals/bioplastics or similar biomaterials.

One pillar embodiment of the invention involves the generation of plant-based bovine food proteins from sustainable nutrients. This is described below where the inputs to the process would be the available grasses, straw, weeds and crop residues obtained from local agriculture in the vicinity of the fermentation installation without the reliance on a monoculture of sugar beet/cane or maize/grain as is currently the case. This TPH embodiment where the current invention is coupled to a biomass fermentation plant designed to generate beef protein, milk protein and/or fermented leather (for example) represents an optimised replacement for dairy and beef farming. In this regard, the protein fermentation processes that aim to generate replacements for milk products and meat and collagen etc currently rely on refined sugar (typically glucose and sucrose) inputs allied to supplementation of the fermentation reactor with external sources of lipids, essential amino acids and trace nutrients and trace elements, some of which may have to be shipped over long distances as may be the case for sugars extracted from sugarcane in the tropics.

In the biofuel/alcohol embodiment, as per FIG. 3, the TPH technology provides superior performance and efficiency at scale compared with existing enzymatic, extruder and steam explosion technologies as regards efficiency of sugar extraction ahead of conversion of the sugars into alcohols and other biofuels such as aviation kerosene. Similarly, the use of sugars extracted from sustainable lignocellulose biomass in the production of bioplastics will be critical to the growth of this new industry where the primary bioplastics currently in production (PLA & PHA) are derived from starch extracted glucose. Other microbial processes that rely on the supply of sustainable nutrient/sugar inputs are also envisaged as being suitable for incorporation of the TPH technology such as glycols and polyesters as described above.

### BRIEF DESCRIPTION OF THE DRAWINGS

How the invention may be put into effect will now be described, by way of example only, with reference to the accompanying drawings, in which:

FIG. 1 is a block diagram of apparatus for the TPH processing of agricultural crop residues and lignocellulosic biomass, the production of protein by fermentation allied to the production of biogas to generate energy for the process;

FIG. 2 is a view in longitudinal section of a rotary TPH vessel forming part of the apparatus of FIG. 1;

FIG. 3 is a plot of % hydrolysis efficiency as measured by BMP against fermentation time in days for straw treated using milling, existing prior art treatment, TPH and alkali TPH; and

FIG. 4 shows the relationship between sodium hydroxide dosage added to straw in a single or two-step process for alkali TPH pre-treatment and the BMP of hydrolysate during subsequent enzymic degradation.

### DETAILED DESCRIPTION OF PREFERRED EMBODIMENTS

As is well described, plants form two primary types of cell wall that differ in function and composition. Primary cell walls surround growing and dividing plant cells. These provide mechanical strength but must also expand to allow the cell to grow and divide. A much thicker and stronger secondary wall accounts for most of the carbohydrate present in biomass and is deposited once the cell has ceased to grow. The secondary walls of wood and grasses are composed predominantly of cellulose, lignin, and hemicellulose (xylan, glucuronoxylan, arabinoxylan, or glucomannan). These are referred to as structural carbohydrates. Cellulose fibrils are embedded in a network of hemicellulose and lignin. Cross-linking of this network results in the elimination of water from the wall and this arrangement is a major contributor to the structural characteristics of secondary walls. The structure also forms a waxy hydrophobic composite that limits accessibility to enzymes. Those enzymes that are excluded include those involved in biodegradation in nature and also include the enzymes generated by microbes during both aerobic and anaerobic microbial fermentation or digestion in industrial bioreactors.

The TPH process using saturated steam causes cellular disruption of lignocellulosic cell walls that disrupts the bonds between the protective lignin and the underlying cellulose and hemicellulose. The cellular disruption also releases soluble sugars present and the process generates organic acids. Furthermore, the process initiates the biochemical cleavage of the cellulose and hemicellulose into soluble sugars, i.e. glucose and xylose respectively. This process is enhanced in an alkaline environment where alkali is added to the substrate for a soak period prior to loading to the TPH vessel and/or added directly to the TPH vessel to facilitate a high pH/high temperature hydrolysis. This combined process facilitates subsequent fermentation and/or digestion in downstream bioreactors and in the case of a naturally buoyant low-density biomass such as straw, this TPH process causes the material to be become non-buoyant and hence compatible with subsequent aqueous fermentation or digestion in a digester in which a body of aqueous liquid in which digestion/fermentation is taking place is contained in a stirred reactor under optimum temperature conditions for the target microbial species and/or enzymes. These temperatures are typically above ambient where the microbial biomass is maintained at either mesophilic or thermophilic ranges where external heat is required. Oxygen/air may also be required to be injected into the reactor for strictly aerobic microbial species.

In FIG. 1, lignocellulosic agricultural biomass/crop residues 12 recovered from cultivated land 10 are passed from shredder/mixer 14 to a rotary TPH autoclave 36 as described in WO 2012/172329 (Toll et al., the disclosure of which is incorporated herein by reference). An alkali in the form of sodium hydroxide, magnesium hydroxide, calcium hydroxide and/or potassium hydroxide alone or as a mixture is stored in a hopper/tank 13 and the alkali(s) are added to the batch as a solid or liquid solution prior to loading or during loading. The alkaline hydroxide is mixed through the biomass to increase the pH and alkalinity of the material ahead of process initiation. Other alkalis can also potentially be used. A blend of the above alkalis is typically applied where the ratio relates to the hydrolytic performance, the downstream requirements of the biological processes and the final products as regards the anions used. Following alkaline steam treatment, the resulting hydrolysed material is discharged from autoclave 36 to a hopper or other container 16. From here the hydrolysate can be processed further as per the following scenarios that depend on the downstream fermentation requirement:
(a) The whole hydrolysate is passed directly to the saccharification reactor 20 without screening or centrifugation. This may involve a neutralization step.
(b) The hydrolysate is fed to a separator 18 which may be a centrifuge and/or filter that is configured depending on the down-stream fermentation/digestion requirements. In this embodiment the fibre is fed to the saccharification reactor, and the centrate/filtrate is returned to the shredder/mixer 14 for reuse. Rinsing or neutralisation may be applied to reduce the pH and/or recover excess alkali.
(c) The filtrate is passed to the saccharification reactor with the fibres on-passed to the biogas reactor 32 where this material undergoes mesophilic or thermophilic wet anaerobic digestion for the purposes of biogas production and stabilization of the residual excess biomass fibre.

As explained above, an adjustment in the pH of the hydrolysate may be required in some applications depending on the final pH of the hydrolysate that typically falls during TPH treatment and as will be required by downstream enzymatic and microbial processes.

From the biogas reactor 32, a stream of biogas is supplied to a combined heat and power (CHP) plant (and/or boiler) 37 which supplies renewal electricity for carrying out various process steps. Renewable heat is also generated for various process steps including, in particular, the generation of steam for the autoclave 36 as disclosed in WO 2012/172329, in addition to maintaining the target temperatures in the fermentation and biogas reactors. External sources of heat are also envisaged such as the utilization of a proportion of the biomass as fuel to a biomass boiler where the parasitic demand is expected to be <10% of the total biomass used. Digestate 34 from the biogas reactor 32 may be used as a soil conditioner/peat replacement product and may be preferentially returned to the cultivated land 10 to support subsequent harvests of biomass for the process. Following the completion of saccharification within the reactor 20, the hydrolysate passes to the fermentation reactor 22. It is also possible in some embodiments that reactors 20 and 22 are combined to facilitate parallel saccharification and fermentation processes in the same vessel. As per the illustrated embodiment, the biomass protein from the selected microbial species then passes to a protein refiner 24, from which a protein product stream 26 is recovered and a residue stream is passed out as waste biomass 28 for further recovery within the TPH process to optimise sugar extraction efficiencies and/or to generate further energy in the form of biogas for the overall fermentation facility by passage to the biogas reactor 32.

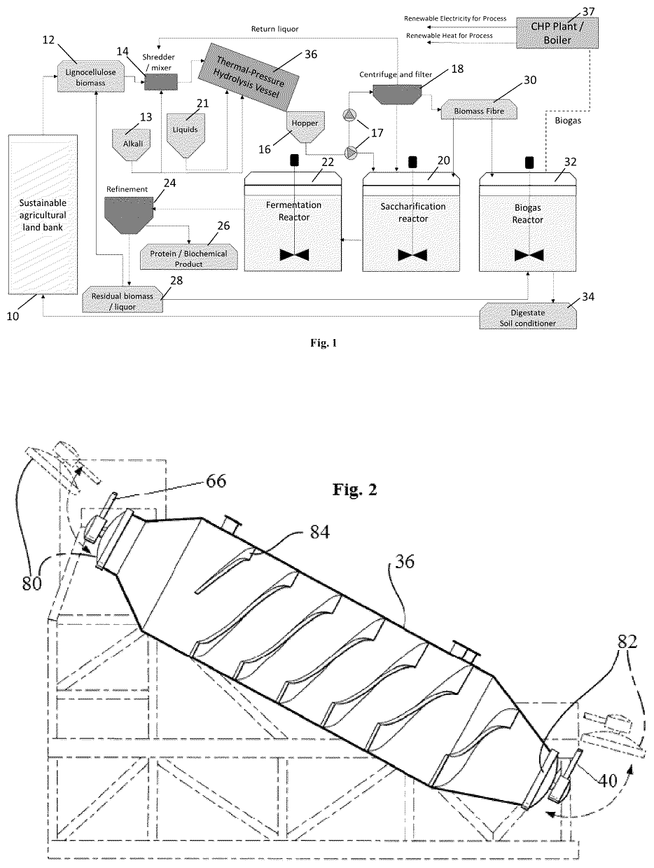

A representative rotary pressure vessel 36 described in some detail in WO 2012/172329 (Toll et al.), is shown diagrammatically in FIG. 2 and is provided with inlet and discharge doors 80, 82 respectively connected to lines 40 and 66 and internal flights 84. If the pressure vessel is rotated in one direction the flights 84 in addition to circulating the feedstock forwards towards the lower discharge end and when rotated in the other direction lifts the material from the lower end while maintaining circulation of the material. On completion of a vacuum pre-treatment stage, which may last about 15 minutes, steam and optionally water are introduced through door 82 to raise the internal temperature of the pressure vessel e.g., typically to about 140-160° C. and to about 4-6 bar. This may vary between 11° and 180° C. depending on the biomass type being processed and the concentration of alkali applied to each batch. Pressurization of the TPH vessel may take some minutes, quantities of the introduced steam condensing in the initially relatively cold load to increase the water content thereof. Circulation of the load through the TPH vessel by reverse rotation may be carried out, and even load distribution may be monitored by transducers measuring weight at upper and lower ends of the rotary vessel to check that the load has not compacted and remains at the bottom of the vessel. Penetration of the steam into and through the load is gradual, and pressure is monitored at both ends of the TPH vessel, rise of pressure at the upper end of the TPH vessel to or close to the rated processing temperature 110-180° C. indicating that the pressurization step is complete. By introducing steam from the lower end and monitoring pressure (or temperature) at the upper end of the vessel, it is possible to ensure that the whole of the load has been penetrated by the steam. Processing at the working temperature and pressure is then carried out for a period of time effective to break down the load and in particular the lignocellulosic content thereof.

The feedstock may be tumbling in an atmosphere of wet steam in pressure vessel 36 at >2-8 bar and at >110-180° C. e.g., about 140-160° C. and about 4-6 bar at variable alkalinities. The pressure vessel 36 which may be downwardly inclined e.g., at about 15° has an insulating jacket to reduce unwanted heat loss but is heated solely internally and solely by wet steam from a steam accumulator introduced into its lower end via line 40. Because the atmosphere is of wet steam, the interior surface of the pressure vessel is covered with a thin layer of liquid water, and unwanted adhesion of organic material is not promoted. The steam in the accumulator is generated using CHP plant 37 fed with biogas in line from anaerobic digestion and if necessary, also with auxiliary fuel via a gas, biofuel or from a steam biomass boiler where a proportion of the dry lignocellulosic biomass is used at a rate of <10% of that supplied to the TPH. In the FIG. 2 embodiment the pressure vessel 36 is rotary and is, as previously explained, is provided with internal flights 84 for circulating the feedstock. Treatment times will depend on the biomass type and on the temperature and alkalinity employed, but in some embodiments may be 20-60 minutes.

The TPH process promotes the chemical breakdown of lignocellulose materials. Specifically, this hydrolysis results in:
(a) cellular disruption to release existing soluble cellular sugars and hydrolyse starch,
(b) the delignification of the biomass to release the underlying cellulose and hemicelluloses to downstream saccharification and
(c) the cleavage of chemical bonds in the presence of water as steam where cellulose is partially converted to C₆ sugars, e.g., glucose, and hemicellulose is more substantially converted to C₅ sugars e.g. xylose.

The natural equivalent of this process in the production of animal protein is the enzymatic conversion of grass to sugars within ruminant's digestive tract. Literature data suggests that mechanical and enzymatic extraction of sugar from various grasses within bovine digestive tracts can convert approx. 20% of the total carbohydrates present into digestible sugars depending on the quality of the grass fed where this digestive process takes several days to achieve. The TPH data demonstrates that 20-30% of the total organic content (volatile solids) of certain grass and hay can be converted to fermentable sugars at approx. 140° C. within less than one hour. Accordingly, the TPH method is at least as effective as in vivo enzymatic hydrolysis of grass and possibly up to 50% more efficient after the initial TPH treatment. However, the TPH process also facilitates the hydrolysis of plant materials that ruminants cannot readily digest effectively or are otherwise unpalatable such as straw that contains only trace amounts of starch and free sugars. For such lignocellulosic material the post-TPH enzymatic saccharification process has the potential to convert up to 93% of the cellulose and hemicellulose present into glucose and xylose that is then bioavailable to microbes for metabolic processes.

On completion of TPH treatment, the saturated steam in the pressure vessel 36 may be condensed by depressurization to reduce the internal temperature below 100° C. In this regard the atmosphere may be vented, e.g., through line 66 from the upper end of the pressure vessel, opposite to where steam is introduced. Steam in line 66 may be used to pre-heat the contents of the liquid tank 21; in addition, or as an alternative it may be passed to the steam condenser. After the filling stage of the hydrolysis cycle, the line 66 may also be used for evacuation of air in the vessel 36.

Hydrolysed biomass from the TPH vessel is discharged from its lower end to a discharge tank or hopper 16 where its moisture content may be further adjusted and mixed. It may be cooled by a heat exchanger that is thermostatically controlled to achieve a precise temperature of the output entering the separator 18 or the saccharification tank 20 ahead of transfer to the fermentation reactor 22 where heat shock could potentially inhibit the subsequent fermentation processes.

Essentially the TPH technology coupled to a fermentation reactor plant replaces the bovine model of protein production. Therefore, as per the drawings, the initial milling and shredding of grass silage and other crop materials mixed with recycled microbial biomass represents the initial “chewing of the cud” where the subsequent improved efficiency of the thermal pressure hydrolysis (TPH) mechanical “rumen” allows the inclusion of lignocellulosic biomass inputs that conventional bovines cannot readily assimilate. This increase in the bandwidth of biomass substrates allied to the greatly increased efficiency of hydrolysis and optimises the land-use efficiency.

It is envisaged that such a facility would also utilize conventional high starch inputs such as sugar beet and corn as sustainably available where these crops would be processed in addition to the other lignocellulose inputs. Given the different temperature requirements of high starch crops such as beet, these would be processed in discrete batches at lower temperatures to the lignocellulose materials that require higher temperatures and alkali addition for optimum sugar extraction.

After mixing the excess wet fermentation biomass with the primary lignocellulose biomass and/or the conventional crop components with additional water as required to optimise the subsequent hydrolysis process, the substrate is fed into the TPH mechanical “rumen” 36. Laboratory tests utilizing a pilot TPH plant demonstrates that the efficiency of sugar extraction from comparable grass inputs (as per the staple diet of bovines) is equivalent to or up to 50% more efficient than the enzymatic processes in the rumen while hydrolysis efficiencies of 85-95% in the subsequent saccharification reactor is possible in the case of inputs not normally fed to cattle such as straw where the base point digestibility is therefore zero.

In the case of the typical 64 m³ TPH vessel deployment, this has an approx. capacity of 150 fresh weight tonnes per day of grass silage at a dry matter (DM) of 25-32%. At a standardised 30% DM, this represents a capacity of 45 dry tonnes per day. In the case of straw where the DM is approx. 90%, the dry tonnage throughput per day will be the same. This then equates to 50 fresh weight tonnes per day of straw. Cattle typically consume up to 30 kg fresh weight of grass per day. Therefore, the 150 tonnes per day TPH capacity represents the consumption of approx. 5,000 cattle/cows. Adding then the initial approx. 0-50% uplift in sugar extraction from grass, the yield of sugar per TPH unit can be equivalent to between 5,000-7,500 bovine units. When the downstream saccharification step of the delignified cellulose and hemicellulose is added this equates overall to a maximum 95% saccharification efficiency. Therefore, a single TPH can be expected to represent the sugar output that is >200% more efficient than the bovine model based on grass where the sugar output of a single TPH would represent >10,000 bovine units. Further to this, with the inclusion of normally indigestible lignocellulose inputs to the TPH process such as crop residues and straw etc to supplement the grass, the system will be a substantially more efficient process in converting available biomass within a given geographical land area into sugars and other soluble nutrients where these sugars and nutrients are then used as the primary feedstock within fermentation reactors to generate bovine products such as milk, meat and leather proteins etc.

The approx. 200%+ improvement in sugar productivity already represents an equivalent reduction in land use for the same protein output. Where, other lignocellulosic crop residues generated from conventional plant-based agriculture are used to supplement the primary grass input as available, the land efficiency will increase further. Furthermore, as the conversion efficiencies of sugar to protein in fermentation reactors can be 8-10 times more efficient than the bovine model, this TPH-fermentation model has the potential to be >20 times more land efficient per unit protein output while avoiding additional tillage and monocultures associated with conventional sugar production, i.e., the TPH-fermentation model has the potential to produce the same protein production as cows with <5% of the land. Furthermore, managed grass lands contribute significantly less greenhouse gas emissions than equivalent tillage land where the high emissions of CO₂ from ploughing are avoided. Emissions from the transport of biomass is also minimised as the inputs can be sourced locally over much shorter distances given the efficiencies involved without significant changes to land-use other than the ultimate displacement of beef and dairy herds. This model can be applied to other protein fermentation processes that seek to displace other animal-based systems such as poultry, pigs and even fish. In the case of fish protein, the TPH of sustainable macro algae biomass in addition to terrestrial biomass to release sugars for the production of replacement finfish and shellfish protein through fermentation is envisaged, and thus protect marine ecosystems as well as preserving terrestrial ecosystems.

After the 2.5-3.0 hours of TPH treatment, the hydrolysed and sterilized substrate is discharged to the buffer tank/hopper 16. Typically, the lignocellulosic biomass entering the TPH vessel 36 will be adjusted to approx. 25-32% dry matter (DM), and after the condensing of the steam injected into the vessel, the hot hydrolysed substrate exiting the vessel will have a DM of approx. 18-22%. After initial de-stoning/contamination removal, as required, this hot substrate is then cooled via passage through a heat exchanger that also pre-heats the dilution water for subsequent batches while cooling the hydrolysed biomass. This fluid is then further size reduced by passage through maceration pumps 17 where the softening of the lignin/cellulose structures during the TPH process makes the residual fibre more amenable to wet milling than the raw materials. This finely divided slurry is then presented sequentially to bespoke centrifuges, screens, and filtration/refinement equipment 18 to separate out the required soluble nutrient/sugar fractions and/or the delignified fibre for on-pass to the downstream protein fermentation and biogas processes. This centrate may be further filtered to generate the required growth medium for the downstream protein fermentation processes to match the process and microbial species-specific requirements of the respective fermentation processes as regards the required sugars, amino acids and trace nutrients and elements required by the microbial fermentation. This growth medium may then be further fortified and enhanced as required for the specifics of the target fermentation process and product characteristics. These supplements may be added prior to TPH treatment, after separation or directly to the respective reactors 20, 22 & 32. Full separation and refinement of the sugars is also possible between reactors 20 & 22 as may be required by other downstream processes such as bioplastic fermentation using similar techniques as used by the sugar industry and or fractionation to remove dissolved impurities or potentially toxic substances.

In all cases the fibre and other fractions that are not utilized by the fermentation process or remain after fermentation 28 can be reconstituted as a pumpable slurry and are available to be transferred to an anaerobic reactor 32 for the purposes of biogas production as per GB 2477423 (Toll), WO 2012/172329 (Toll et al), U.S. Pat. No. 10,907,303 (Toll) and WO 2012/172329 (Blondin). In this regard, bio-available organic materials are expected to remain in the fibre after sugar extraction. Therefore, this material will have a viable biogas potential where the biogas is then used by on-site within combined heat and power (CHP) plants and boilers to generate a proportion of the required renewable energy for the overall facility. This would include steam generation for the TPH process, low grade heat for the fermentation reactors and anaerobic digesters with electricity for plant elements that will greatly improve the carbon efficiency of the overall fermentation plant process.

At the end of the fermentation process after the active biomass and/or sugar, protein and/or the biochemicals are extracted for the generation of the various bovine replacement products such as milk and meat replacement products, any waste liquors and biomass 28 can be recycled to the TPH infeed for further sugar and nutrient recovery and recycling. This can be managed on a batch basis where the residual materials are deemed unsuitable for reprocessing to the fermenter(s). In such event, the hydrolysed output will be transferred directly to the anaerobic digester(s) for energy recovery and stabilization. In the alternative, the capacity to reprocess excess biomass will allow optimum sugar and nutrient extraction from the biomass while optimising energy efficiencies while eliminating waste. It is also envisaged that the residual biomass 28 may be dried further and combusted in a boiler 37 to provide the heat required by the process.

Therefore, in this embodiment, this >10,000 bovine equivalent unit is capable of generating bovine proteins from a greatly reduced land area (<5%), with a greatly reduced emissions profile while providing the biosecurity required as regards substrate sterilization and being energy self-sufficient. Also, the final side stream output from the biogas reactor 32 will be a sanitized and stable, high quality soil conditioner and/or peat replacement product that can be used to fertilize the local grass leys and crops used to service the fermentation facility or replace peat while providing for carbon sequestration of the residual organic matter not utilized in the process. The incorporation of this invention into a protein, biomaterial or bioplastic fermentation facility will make the protein fermentation strongly carbon negative relative to current petroleum models or even the first-generation protein fermentation model that is reliant on an input of refined sugars from remote monocultures while depending on external energy sources.

A plurality of TPH vessels, fermentation reactors and biogas reactors are envisaged for large-scale deployment of the technology for the protein production levels that will be required to displace and/or substitute for animal agriculture.

The general idea of producing edible protein-containing substances wherein the protein possesses an amino acid profile which, in broad outline meets the specification for essential amino acids as set out in the recommendation of the Food and Agriculture Organisation (United Nations) “Protein Requirements” published 1965, by incubating and proliferating, under aerobic conditions, an organism which is a non-toxic strain of microfungus of the class Fungi Imperfecti was disclosed in GB 1210356 (Arnold et al., Rank Hovis McDougall). However, that specification discloses no specific fungal genus or strains and includes no working example. GB 1331471 (Solomons et al., Rank Hovis McDougall) discloses incubating in a substrate of vegetable origin e.g. wheat feed, hydrolysed potato, molasses, bagasse waste and/or citrus waste with a non-toxic strain of Penicillium notatum or Penicillium chrysogenum. GB 1346062 (also Solomons et al., Rank Hovis McDougall) describes a process for the production of an edible protein-containing substance which comprises incubating and proliferating, under aerobic conditions, a non-toxic strain of the genus Fusarium or a variant or mutant thereof, e.g. Fusarium graminearum (now re-classified as Fusarium venenatum) in a culture medium containing essential growth-promoting nutrient substances, of which carbon in the form of assimilable carbohydrate/sugar constitutes the limiting substrate in proliferation, and separating the proliferated organism comprising the edible protein-containing substance, see also GB-A-2137226 (Marsh, Rank Hovis McDougall), and EP-A-0123434 (also Marsh). Disclosed substrates for the incubation stage include starch, starch containing materials or products of their hydrolysis, glucose, sucrose, sucrose containing materials or hydrolysed sucrose e.g., hydrolysed potato, molasses, maltose, hydrolysed bean starch or cassava.

Biomass rich in protein for human consumption generated through a myco-fermentation process is commercially available under the trade name Quorn™. An alternative process using fungal cells of the order Mucorales is disclosed in WO 01/67886 (Bul et al., DSM NV).

Currently it has been reported that Unilever is partnering with food-tech company Enough (formerly 3F BIO) to bolster its plant-based strategy by tapping into technology that uses a zero-waste fermentation process to grow a high-quality protein. Natural fungi are fed with starch-based biomass feedstock, such as wheat and corn, to produce Abunda mycoprotein, that is marketed as a complete food ingredient containing all essential amino acids and high in dietary fibre. Pegged as a “game-changing” protein, Abunda is stated as being a natural fit for Unilever's fast-growing meat-alternative brand. The Vegetarian Butcher, which saw a 70% growth in 2020. This uses a diverse blend of plant-based proteins to create meat-like tastes and textures for its wide-ranging portfolio.

It has also been reported that Unilever has also partnered with biotech company Algenuity to explore the use of microalgae, where they claim that microalgae is another highly nutritious and sustainable protein powerhouse that can be fermented into a wealth of products such as mayonnaise, soups, sauces and meat alternatives, see e.g. EP-A-3884036 (Spicer et al.).

In principle, any of the above-described organisms might be used in the fermentation reactor 22. Other fermentation routes may be employed to produce e.g. biofuel or bio-plastics e.g., PLA as disclosed in EP-A-2831291 (Forgacs), EP-A-3295754 (Marga), EP-A-3473647 (Dai), EP-A-3452644 (Lee), EP-A-3684800 (Dai), EP-A-3747901 (Purcell) and WO 2004/057008 (Botelo).

In the biofuel/alcohol embodiment, as per FIG. 3, the TPH technology provides superior performance at scale compared with existing mechanical, enzymatic, extruder and steam explosion technologies as regards efficiency of sugar extraction ahead of conversion of the sugars into alcohols. Other microbial processes that rely on the supply of sustainable nutrient inputs are also envisaged as being suitable for incorporation of the TPH technology.

How the invention may be put into effect will now be described in the following examples.

### Example 1

Reducing Sugar Release from Haylage Following Thermal Hydrolysis

An experiment was conducted using a 1 m³ pilot scale TPH vessel that had previously been successfully demonstrated to provide accurate projections as to the performance of the full-scale TPH vessel (64 m³; FIG. 2). This trial was conducted to determine to what extent the TPH process would release reducing sugars from haylage. The haylage was obtained from a baled supply harvested in Dorset during the late summer period. The process followed the same procedure and utilized the same equipment as described in WO 2012/172329 (Toll et al.,) except that the temperature of processing for 40 minutes was maintained at 140° C. as opposed to 160° C. as is typically applied to municipal waste materials. Therefore 17 kg of the haylage was processed and the TPH processed haylage was then centrifuged using a Creda Spin Dryer for 20 minutes to recover a liquid fraction. 41.5% of the total mass of the TPH processed haylage was hydrolysed into the liquid fraction. This liquid fraction was then analysed for total reducing sugar contents. The results are listed in Table 1.

TABLE 1
Sugar extraction from haylage hydrolysate

| Items | Result |
|---|---|
| Mass of haylage input, kg | 17 |
| Dry solids (DS) contents of haylage, % | 78.2 |
| Volatile solids (VS) contents of haylage, % of DS | 89.6 |
| Mass of haylage VS input, kg | 11.9 |
| Mass of TPH output, kg | 53 |
| Mass of liquid fraction separated from the | 22 |
| TPH processed haylage, kg | |
| Total sugar contents of the liquid fraction, % | 12 |
| Mass of reducing sugar produced, kg | 2.6 |
| Specific sugar yield, g sugar/g VS of haylage | 0.22 |
| Sugar productivity, kg sugar/t haylage | 153 |

This trial demonstrated that 22% of the volatile solid (VS) content of the haylage can be extracted as reducing sugars after thermal hydrolysis using the TPH technology at 140° C. for 40 minutes. The extraction of sugars from macerated haylage centrate at ambient temperatures was found to be at trace levels.

### Example 2

Reducing Sugar Release from Barley Straw Following Thermal Hydrolysis

The same procedure as in trial No. 1 was repeated for barley straw which typically contains very low concentrations of free reducing sugars where all the carbohydrate present is in the form of cellulose and hemicellulose bound by lignin. In this 140° C. TPH trial, the extractable reducing sugars in the centrate was found to be at trace levels, i.e., <1%.

### Example 3

### Determination of the Bioavailability of Lignocellulosic Carbohydrate to Enzymatic Degradation Following Thermal Hydrolysis in the Presence and Absence of an Alkaline Hydroxide

Given that haylage typically has a higher free sugar content than straw as reflective of the relative nutritional value to ruminants and the results of Examples 1 and No. 2, the effect of thermal hydrolysis on the subsequent enzymatic conversion of the lignocellulose present into bioavailable sugars was assessed. In this regard, it had already been determined that the bioavailability of thermally hydrolysed straw can be improved by TPH treatment as demonstrated in GB 2546243 (Toll et al.) where the biomethane production from hydrolysed barley straw using a standard 30-day biomethane potential (BMP) test was demonstrated to increase lignocellulosic carbohydrate availability by 32% relative to mechanically milled straw. Based on a theoretical maximum methane yield of barley straw of 480 m³ CH₄/t VS, the biomethane productivity as described increased this observed enzymatic bioavailability from 77% to 81%. Alkaline treatment of lignocellulosic biomass is known from the literature to improve the release of cellulose and hemicellulose from its protective lignin sheath and therefore, a trial was conducted where the straw was initially soaked in a 5% solution of NaOH for 24 hours at room temperature. The straw was then screened and rinsed in water followed by TPH treatment as per the method described in Example 1. This resulted in an increase in the bioavailability of the lignocellulose to 93%, i.e., a 30-day BMP of 448 m³ CH₄/t VS. A test on the wash water demonstrated negligible bioavailable carbohydrate present in this fraction which was consistent with Example 2 without alkali addition.

### Example 4

### Bioavailability of Structural Carbohydrate to Enzymatic Degradation Following Thermal Hydrolysis as a Function of Alkali Concentration.

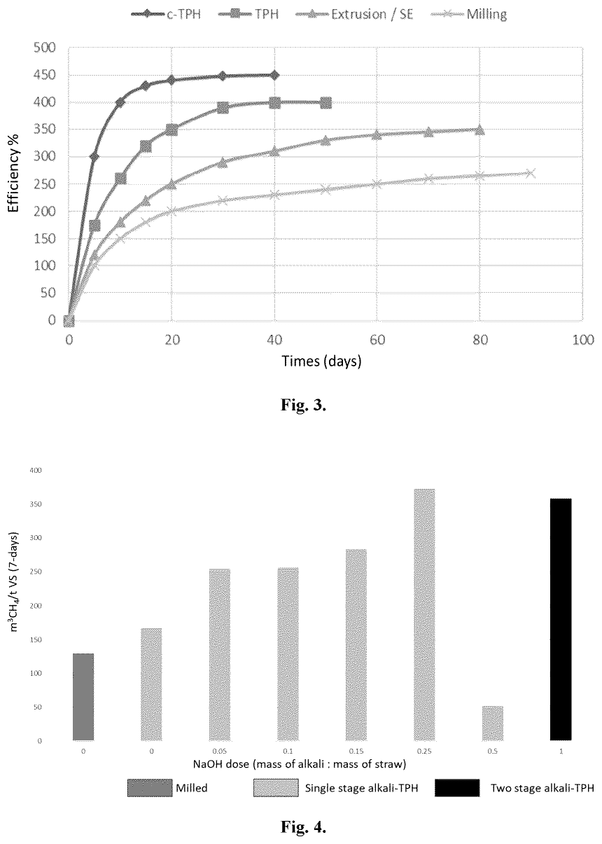

Further to the substantively improved bioavailability of straw lignocellulose to enzymatic degradation following the two-stage process of alkali pre-treatment followed by TPH treatment, a series of tests was conducted to determine if the same very high bioavailability efficiency could be achieved in a single step by conducting the TPH process at high pH. Therefore, the same experimental protocol was followed as per Example 1 where the barley straw was hydrolysed in discrete batches at 140° C. in the presence of increasing concentrations of NaOH within the TPH vessel itself followed by shorter 7-day BMP tests. These results are illustrated in FIG. 4 where the 7-day BMP for the single stage trials are compared with the 7-day result for the two-stage process from Example 3 and with the 7-day BMP for mechanically milled straw. These results demonstrate that processing 1.0 kg straw at 140° C. in the presence of 0.25 kg NaOH resulted in a slightly higher methane yield than the two-stage process, i.e., 374 m³ CH₄/t VS versus 359 m³ CH₄/t VS after 7 days where this was projected to represent a >95% bioavailability of the structural carbohydrate to enzymatic degradation. Even at the lowest concentration tested, i.e., 0.05 kg NaOH to 1.0 kg of straw the BMP of 255 m³ CH₄/t VS was 96% higher than the mechanically milled straw after 7-days.

### REFERENCES

Aski, A. L., Borghci, A., Zenouzi, A., Ashrafi, N. & Taherzadeh, M. J. (2019). Effect of steam explosion on the structural modification of rice straw for enhanced biodegradation and biogas production. BioResources. 14 (1), p 464-485.
Larsen, J, Ostergaard, M & Thirup, L. (2012). Inbicon makes lignocellulosic ethanol a commercial reality. Biomass and Bioenergy. 46, p 436-45.
Sambusiti, C., Monlau, F., Ficara, E., Carrere, H. & Malpci, F. (2013). A comparison of different pre-treatments to increase methane production from two agricultural substrates. Applied Energy. 104 p 62-70.

1. A method of producing an incubatable feedstock for fermentation comprising:
subjecting plant biomass to thermal pressure hydrolysis in an autoclave using steam, agitation and an alkali catalyst;
recovering the hydrolysed product from the TPH vessel;
feeding the recovered hydrolysed product to a separator or directly to a saccharification reactor;
recovering a first separated stream of residual biomass fibres from the separator for biogas production or for saccharification; and
recovering a second separated stream from the separator that is used either as an incubatable feedstock fed to the saccharification reactor or recirculated to the TPH vessel.

2. The method of claim 1, wherein the plant biomass is an agricultural product.

3. The method of claim 1, wherein the plant biomass is a lignocellulose plant material or by-product such as grass or straw or other crop residue.

4. The method of claim 1, wherein the plant biomass comprises straw from barley, oats, rape, rice, rye and/or wheat or a mixture thereof.

5. The method of claim 1, wherein the plant biomass is a fibrous lignocellulosic biomass which is subjected to “TPH-fermentation” by a method which comprises:
introducing a prepared feed batch into a pressure vessel that may include pelletising low-density lignocellulosic biomass to maximise loading per batch;
adding liquid (water and/or organic slurry) to said feed batch; adjusting the alkalinity depending on the severity of treatment required;
introducing an atmosphere of saturated steam into the pressure vessel and maintaining said atmosphere at 110-180° C. and 2-8 bar whilst circulating the material of the alkaline feed batch through the saturated steam atmosphere for a time effective to induce internal collapse and delignification of the lignocellulose biomass;
gradually depressurizing the pressure vessel and cooling its contents; and
recovering the hydrolysed lignocellulose biomass from the pressure vessel as a slurry/sludge in a sterilized state, having been delignified and with a disrupted cellular structure as a result of thermal pressure hydrolysis.

6. The method of claim 1, wherein the pressure vessel has any of the following features:
(a) inlet and discharge ends and a downward incline towards its discharge end;
(b) it is rotary and is provided with helical internal flights for circulating the material of the feed batch through the saturated steam atmosphere;
(c) an internally stirrer with rotary blades or paddles for circulating the material of the feed batch through the saturated steam atmosphere.

7. The method of claim 6, having any of the following features:
(a) the pressure vessel is evacuated between introduction of the feed batch and introduction of saturated steam;
(b) the steam is introduced directly from a boiler or from a steam accumulator;
(c) thermal pressure hydrolysis is at 2-8 bar and at >110-180° C.;
(d) thermal pressure hydrolysis is at about 110-120° C. for high starch crops and >130° C. for lignocellulose materials and as adjusted for the alkalinity of the biomass following the addition of alkali;
(e) steam from completion of one cycle of material circulation is used to heat the liquid/slurry to be combined with the straw or the next cycle.

8. The method of claim 1, wherein the plant biomass comprises early season first cut grasses and the hydrolysis is between 120-140° C. under alkaline conditions.

9. The method of claim 1, wherein the plant biomass comprises later grass cuts, hay, straw, stover and leaves and the hydrolysis is at about 130-160° C. under alkaline conditions.

10. The method of claim 1, further comprising supplying the incubatable feedstock to a saccharification reactor to complete the hydrolysis of the cellulose and hemicellulose present to soluble sugars with an overall hydrolytic efficiency of 85-95%.

11. The method of claim 1, where the saccharified sugars and other nutrients released during the high efficiency hydrolysis are used as a substrate by a nontoxic microorganism that can give rise to a protein, bioplastic, biofuel or other biomaterial product to a fermentation reactor and recovering the protein, bioplastic, biofuel or other biomaterial product therefrom.

12. The method of claim 11, wherein the microorganism is a fungal mycelium, bacteria, archaea, yeast or is microalgae.

13. A fermentable feedstock comprising a plant biomass thermal pressure hydrolysis filtrate, the plant biomass preferably comprising an agricultural crop product with a high starch content such as beet, corn or potato etc, e.g. a lignocellulose plant material such as grass or straw or other crop residue such as straw from barley, oats, rape, rice, rye and wheat or a mixture thereof.

14. A method of producing an incubatable feedstock for fermentation comprising:
subjecting plant biomass to thermal pressure hydrolysis in an autoclave using steam and agitation; recovering the hydrolysed product from the autoclave; and
removing solids from the hydrolysed product to recover a liquid phase which is a sugar/nutrient solution and provides the incubatable feedstock.

15. The method of claim 14 having any of the following:
(i) the plant biomass is a fibrous primary lignocellulose biomass which is subjected to to fermentation by a method which comprises:
introducing a feed batch into a pressure vessel;
adding water and/or organic slurry to said feed batch;
introducing an atmosphere of saturated steam into the pressure vessel and maintaining said atmosphere at 110-180° C. and 2-8 bar whilst circulating the material of the feed batch through the saturated steam atmosphere for a time effective to induce internal collapse of the lignocellulose biomass;
gradually depressurizing the pressure vessel and cooling its contents; and recovering the hydrolyzed lignocellulose biomass from the pressure vessel as a slurry/sludge in a sterilized state, with a disrupted cellular structure as a result of thermal pressure hydrolysis;
(ii) the pressure vessel has any of the following features:
(a) inlet and discharge ends and a downward incline towards its discharge end;
(b) it is rotary and is provided with helical internal flights for circulating the material of the feed batch through the saturated steam atmosphere;
(c) an internally stirrer with rotary blades or paddles for circulating the material of the feed batch through the saturated steam atmosphere;
(iii) any of the following features:
(a) the pressure vessel is evacuated between introduction of the feed batch and introduction of saturated steam;
(b) the steam is introduced from a steam accumulator; (c) thermal pressure hydrolysis is at 2-8 bar and at >110-180° C.;
(d) thermal pressure hydrolysis is at about 110-120° C. for high starch crops and >130° C. for lignocellulose materials;
(e) steam from completion of one cycle of material circulation is used to heat the slurry to be combined with the straw or the next cycle.
